(19) United States
(12) Patent Application Publication
(10) Pub. No.: US 2004/0268429 A1
(43) Pub. Date: Dec. 30, 2004

(54) Rapid and efficient micropropagation system for Copihue (Lapageria rosea)

(76) Inventors: Manuel Gidekel, Temuco (CL); Isabel Vallandares, Temuco (CL); Yessica Rivas, Temuco (CL); Ana Gutierrez, Temuco (CL); Luis Destefano Beltran, Fargo, ND (US); Emilio Guerra, Temuco (CL)

Correspondence Address: John Dodds, 1707 N St. NW, Washington, DC, 20036, US

(21) Appl. No.: 10/857,994

(22) Filed: Jun. 1, 2004

Related U.S. Application Data
(60) Provisional application No. 60/475,255, filed on Jun. 3, 2003.

(51) Int. Cl. A01H 1/00; A01H 1/02

(52) U.S. Cl. 800/260

(57) ABSTRACT

A rapid micro propagation system of Copihue plants is disclosed here. The propagation according to the present system uses apical meristems of at least seven years old plants as the intiation material. According to the present system vigorous and healthy plantlets with predictable features can be obtained without callus formation in about five months. The method is applicable to a vast number of different Copihue cultivars.

### CROSS REFERENCE TO RELATED APPLICATIONS

This non provisional patent application claims priority date of the U.S. provisional patent application No. 60/475,255 filed on Jun. 3^{rd}, 2003.

### FEDERAL SUPPORT

This invention that is the subject matter or the present application was not a recipient of any federal support for its research and development. The research has been funded with two grants of Chilean agencies Fundacion Andes 1364-10 and FIT 001-A1-013 INNOVA BIO BIO.

### BACKGROUND OF THE INVENTION

1. Field of the Invention

The present invention relates to the field of propagation of plant species or varieties and more particularly to micro propagation or in vitro propagation of Lapageria rosea.

2. Background

Copihue (Lapageria rosea) is a monocotyledon liane endemic to Chile. It is also the national flower of Chile. Copihue is a highly prized ornamental. Copihue is a rather unique plant in that it takes seven years for it to flower when grown from a seed. The species is in danger of extinction due to great commercial demand. In order to save the species from extinction and to meet the growing commercial demand a rapid and efficient micropropagation system is needed.

The methods so far known to propagate the species are however, slow or they do not enable production of vigorous plants. Typically the methods so far known are not successful for propagation of several existing Copihue varieties, but may be used to one specific variety. Therefore, there is a high need for a rapid method for propagation of any Copihue variety.

The methods so far known enable multiplication using only young plants not yet able to flower as initiation material. The new plantlets according to any known method therefore need several years before flowering. Furthermore the problem in methods using young plants as initiation material is that the features of the adult plant, including the characteristics of the flowers, is not known. Therefore the methods known before are not very predictable.

An efficient micropropagation system is also needed to accelerate application of genetic engineering techniques for genetic improvement of Lapageria rosea species.

The micropropagation method according to the present invention resolves the problems related to Copihue propagation and it provides a rapid multiplication system to provide Copihue plants of good quality and vigor.

### SUMMARY OF INVENTION

The object of the present invention therefore is to provide a predictable and rapid method to multiply a large number of Copihue varieties.

The method according to the present invention provides Copihue plants that behave like adult plants, i.e. they flower when they are about 1 meter of height and do not need several years of maturation.

With the micropropagation system described in this disclosure rooted and vigorous Copihue plantlets can be produced in just 5 to 6 moths without any callus stage.

An advantage of skipping callus stage is that somaclonal variation is minimized or even fully prevented, whereby the vegetative clone produced is more likely similar to the mother plants. Therefore the method according to the present invention is clearly more predictable as any of the earlier known method. Furthermore, organogenesis becomes faster and better without callus formation.

Unlike earlier systems, this micropropagation system uses meristems from approximately 10-year-old plants. The meristems can be taken from plants older than 7 years.

An advantage of the present system using plants older than 7 years is that the adult characteristics of the mother plant are known and therefore the characteristics of the vegetative clone are predictable.

One object of the present invention is to provide a rapid micropropagation system for Copihue (Lapageria rosea) plants of comparable quality and vigor.

Another object of this invention is to provide a micropropagation system applicable to numerous Copihue varieties, including commercial varieties.

Still another object of the present invention is to provide a micropropagation system of low cost.

Another object of the present invention is to provide Copihue vegetative clones behaving like adult plants that are able to flower.

A still another object of the present invention is to provide a multiplication method for Copihue plants that does not involve callus formation and thereby no somaclonal variation.

A still another object of the present invention is to provide healthy and virus free Copihue plantlets.

A further object of the present invention is to provide a micropropagation system that is easy to implement.

### BRIEF DESCRIPTION OF THE DRAWINGS

FIG. 1. is a schematic illustration of multiplication of Copihue plant according to the present method.

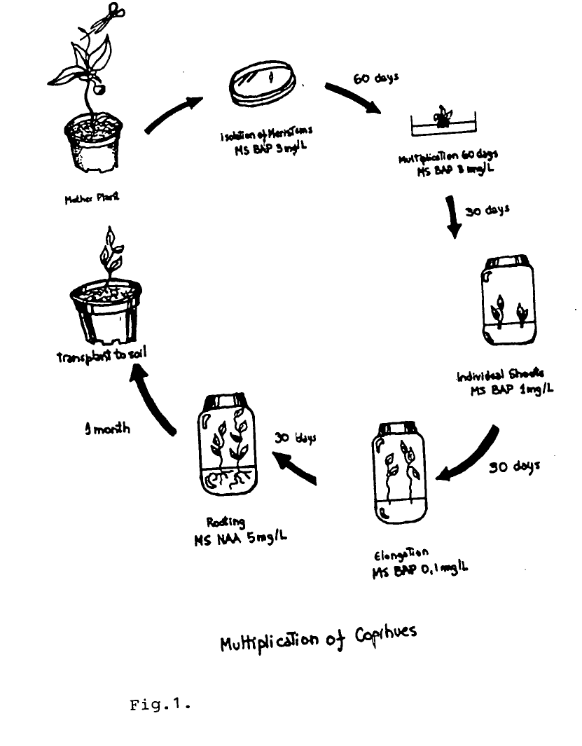

FIG. 2. is a photograph illustrating the steps according to the present propagation method in two different Copihue varieties.

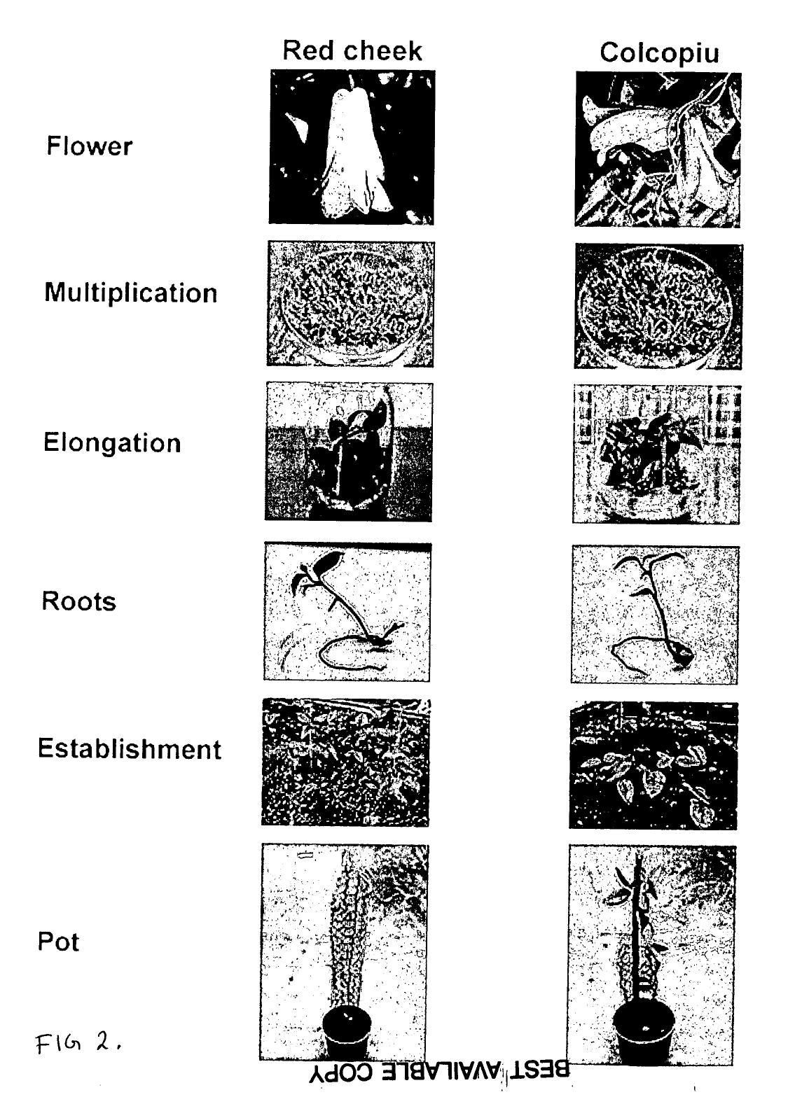

### DETAILED DESCRIPTION OF THE INVENTION

The present invention is directed to micropropagation of several varieties of Copihue (Lapageria rosea) plant.

Micro propagation according to the method of the present invention is carried out with Copihue plant tissue obtained preferably from plants older than 7 years and most preferably 10 years old. The starting material according to the present invention is sterile apical and lateral meristems.

The micropropagation system according to the present disclosure is applicable to at least the following English cultivars: Nash Court, Penheale, Wisley Picotee, Wisley Spotted, Beatrix Anderson, Red Cheek and Flesh Pink.

The micropropagation system according to the present disclosure is applicable to at least the following Chilean cultivars: Nahuelbuta, Colcopiu, Collinge, Remutral, Contulmo, Raimilla, El Vergel, Cobquecura, Rayen, Colibri, Toqui, Caupolican, Malleco

One embodiment of the present invention comprises Murashige Skoog culture medium, pH 5.7, and containing components as defined in Table 1. The culture medium according to the present invention comprises auxin and cytokinine supplements as shown in Table 2.

TABLE 1
Salt components of Murashige Skoog (MS) medium

| | Mg/l |
|---|---|
| Inorganic salts | |
| NH₄NO₃ | 1650.00 |
| H₃BO₃ | 6.20 |
| CaCl₂ | 332.20 |
| CuSO₄ | 0.01 |
| Na₂ EDTA | 0.02 |
| FeSO₄—7H₂O | 37.25 |
| MgSO₄ | 27.80 |
| MnSO₄—H₂O | 16.90 |
| Na₂MoO₄—2H₂O | 0.25 |
| KI | 0.83 |
| KNO₃ | 1900.00 |
| KH₂PO₄ | 170.00 |
| ZnSO₄—H₂O | 5.37 |
| Vitamins | |
| i-inositol | 100 |
| thiamine-HCl | 0.4 |
| Other components | |
| sugars | 30 000 |

TABLE 2
Concentration of auxins and cytokinines in the culture
medium during different stages of plant development.

| Stage | Auxins mg/l | Cytokinines mg/l |
|---|---|---|
| Establishing | n.a. | BAP 3 mg/l |
| Multiplication | n.a. | BAP 1 and 3 mg/l |
| Elongation | n.a | BAP 0.1 mg/l |
| Rooting | NAA 5 mg/l | |

Abbreviations:
BAP N-6-Benzylaminopurine;
NAA naphthaleneaceticacid

It is well known for those skilled in the art, that cultivation mediums are cultivar specific. Therefore, medium applicable to one cultivar does not usually fit for another cultivar. Surprisingly and unexpectedly, according to the present invention the one medium is applicable to a vast number of Copihue cultivars.

Preferred Embodiment of the Invention

Isolated and sterilized apical meristems or lateral meristems obtained from 10 years old Copihue plants are transferred on MS culture medium, pH 5.7, described in Table 1 and being supplemented with 3 mg/l BAP. The meristems are preferably placed on the medium side ways, i.e. parallel to the medium. The meristems are cultured for 60 days where after they give rise to multiple shoots.

After 30 more days of cultivation individual shoots are transferred on MS culture medium containing 1 mg/l BAP. After 30 days cultivation on this medium the plantlets are transferred on MS medium containing 0.1 mg/l BAP. On this medium the shoots will begin to elongate. After 30 days the elongated plantlets are transferred on rooting MS-medium, containing 5 mg/l NAA. The roots will begin to develop and after about one month the plantlets can be transferred to soil. The most preferred conditions are that the plantlets develop by providing a constant the temperature of 22° C. and the lightning with metal halide lamps of 400 W.

While particular embodiments of the present invention have been shown and described, one skilled in the art would realize that changes and modifications may be made without departing from this invention.

What is claimed is:

1. A method for micropropagating vigorous Copihue plants, said method comprising the steps of:
a) isolating and sterilizing apical or lateral meristems obtained from Copihue plants of more than 7 years old;
b) cultivating the sterilized meristems in MS culture medium pH 5.7 supplemented with 3 mg/l BAP;
c) transferring the shoots into MS culture medium pH 5.7 supplemented with 1 mg/l BAP and cultivating them about 30 days;
d) transferring the shoots into MS culture medium pH 5.7 supplemented with 0.1 mg/l BAP and cultivating them until the shoots have elongated;
e) transferring elongated plantlets into MS culture medium pH 5.7 containing 5 mg/l NAA and cultivating them until roots are developed; and
f) transferring rooted plantlets into soil.

2. The method according to claim 1, wherein the meristems are placed sideways on the culture medium.

3. The method according to claim 2 wherein the meristems are obtained from 10 years old Copihue plants.

4. The method according to claim 2 wherein the Copihue plant is selected from the group consisting cultivars Nash Court, Penheale, Wisley Pictee, Wisley Spotted, Beatrix Anderson, Flesh Pink, Red Cheek, Nahuelbuta, Colcopiu, Collinge, Remutral, Contulmo, Raimilla, El Vergel, Cobquecura, Rayen, Colibri, Toqui, Caupolican, Malleco.

5. The method according to claim 2 wherein step b) lasts for about 30 days after the shoots have appeared.

6. The method according to claim 2 wherein step d) lasts for about 30 days.

7. The method according to claim 2, wherein step e) lasts for about 30 days.

8. The method according to claim 2, wherein steps are carried out at constant temperature of 22 C.

9. The method according to claim 2 wherein the Copihue plant is selected from the group consisting cultivars Nash Court, Penheale, Wisley Pictee, Wisley Spotted, Beatrix Anderson, Flesh Pink, Red Cheek, Nahuelbuta, Colcopiu, Collinge, Remutral, Contulmo, Raimilla, El Vergel, Cobquecura, Rayen, Colibri, Toqui, Caupolican, Malleco.

10. A Copihue plantlet propagated according to the method of claim 1.

11. The Copihue plantlet according to claim 10, wherein the plant belongs to cultivar selected from the group consisting cultivars Nash Court, Penheale, Wisley Pictee, Wisley Spotted, Red Cheek, Beatrix Anderson, Flesh Pink, Red Cheek, Nahuelbuta, Colcopiu, Collinge, Remutral, Contulmo, Raimilla, El Vergel, Cobquecura, Rayen, Colibri, Toqui, Caupolican, Malleco.

12. The method according to claim 2, wherein the MS medium comprises 1650 mg l⁻¹ NH₄NO₃, 6.2 mg l⁻¹H₃BO₃, 332.20 mg l⁻¹ CaCl₂, 0.01 mg l⁻¹ CuSO₄, 0.02 mg l⁻¹ Na₂ EDTA, 37.25 FeSO₄H₂O, 27.80 mg l⁻¹ MgSO₄, 16.9 MnSO₄H₂O, 0.25 mg l⁻¹ Na₂ MoO₄2H₂O, 0.83 mg l⁻¹ KI, 1900 mg l⁻¹ KNO₃, 170 mg l⁻¹ KH₂PO₄, 5.37 mg l⁻¹ ZnSO₄H₂O, 100 mg l⁻¹ i-inositol, 0.4 mg l⁻¹ thiamine HCl, and 30 000 mg l⁻¹ sugars.

13. The method according to claim 12. wherein the Copihue plant is selected from the group consisting cultivars Nash Court, Penheale, Wisley Pictee, Wisley Spotted, Beatrix Anderson, Flesh Pink, Red Cheek, Nahuelbuta, Colcopiu, Collinge, Remutral, Contulmo, Raimilla, El Vergel, Cobquecura, Rayen, Colibri, Toqui, Caupolican, Malleco.

14 The method according to claim 12. wherein step b) lasts for about 30 days after the shoots have appeared.

15. The method according to claim 12 wherein step d) lasts for about 30 days.

16. The method according to claim 12, wherein step e) lasts for about 30 days.

17. The method according to claim 12, wherein steps are carried out at constant temperature of 22 C.

18. The method according to claim 12 wherein the Copihue plant is selected from the group consisting cultivars Nash Court, Penheale, Wisley Pictee, Wisley Spotted, Beatrix Anderson, Flesh Pink, Red Cheek, Nahuelbuta, Colcopiu, Collinge, Remutral, Contulmo, Raimilla, El Vergel, Cobquecura, Rayen, Colibri, Toqui, Caupolican, Malleco.

19. A Copihue plantlet propagated according to the method of claim 12.

20. The Copihue plantlet according to claim 19, wherein the plant belongs to cultivar selected from the group consisting cultivars Nash Court, Penheale, Wisley Pictee, Wisley Spotted, Red Cheek, Beatrix Anderson, Flesh Pink, Nahuelbuta, Colcopiu, Collinge, Remutral, Contulmo, Raimilla, El Vergel, Cobquecura, Rayen, Colibri, Toqui, Caupolican, Malleco.
